(19) United States
(12) Patent Application Publication
(10) Pub. No.: US 2021/0192739 A1
(43) Pub. Date: Jun. 24, 2021

(54) COMPUTER-IMPLEMENTED METHOD FOR PROCESSING X-RAY IMAGES

(71) Applicant: Siemens Healthcare GmbH, Erlangen (DE)

(72) Inventor: Stanislav Tashenov, Heroldsbach (DE)

(21) Appl. No.: 17/126,687

(22) Filed: Dec. 18, 2020

(30) Foreign Application Priority Data
Dec. 19, 2019 (DE) 10 2019 220 147.4

(51) Int. Cl. G06T 7/00; A61B 6/00; A61B 6/12; G06T 7/62; G06T 3/40

(52) U.S. Cl. CPC G06T 7/0016; A61B 6/481; A61B 6/504; A61B 6/12; A61B 6/486; G06T 2207/30021; G06T 7/62; G06T 3/40; G06T 2207/10016; G06T 2207/10116; G06T 2207/30101; A61B 6/507

(57) ABSTRACT

A computer-implemented method for processing x-ray images. A sequence of x-ray images is provided of a field of view in which at least one section of a vascular system of an examination subjects is imaged. The sequence describes a variation with time of the detected x-ray intensity in the field of view based on a change in a contrast agent distribution in the section of the vascular system. A contrast agent variable is determined relating to the contrast agent distribution as a function of a variation with time of a variable relating to the x-ray intensity in a selected subregion of the x-ray images. An image of the section of the vascular system is determined as a function of at least one of the x-ray images of the sequence. The image is modified as a function of the contrast agent variable.

### CROSS REFERENCE TO RELATED APPLICATIONS

This patent document also claims the benefit of DE 102019220147.4 filed on Dec. 19, 2019 which is hereby incorporated in its entirety by reference.

### FIELD

Embodiments relate to a computer-implemented method for processing x-ray images.

### BACKGROUND

In the field of contrast-agent-based x-ray imaging, for example within the context of an angiography imaging procedure, blood is rendered visible in x-ray images as a result of an injection of contrast agent into the vascular system of the patient undergoing examination. Contrast media containing iodine or gadolinium may be used as contrast agents, for example. Suitable contrast agents temporarily replace the blood in the respective blood vessel at least partially or even completely. A sequence of x-ray images is acquired during or immediately after injection of the contrast agent. Since the x-ray absorption of suitable contrast agents is significantly higher than the x-ray absorption of the blood or of the surrounding tissue, the part of the vascular system affected by the contrast agent is depicted darker than the background in the individual images.

In order to visualize a larger section of the vascular system, either an image may be selected in which a sufficient amount of contrast agent is present substantially in the entire imaged section of the vascular system or the different x-ray images of the sequence may be combined in such a way that for each picture element or pixel, for example, the value of the pixel is selected for that x-ray image in which the corresponding pixel is presented at its darkest, specifically in which the strongest absorption occurs. This provides the entire section of the vascular system present in the field of view to be clearly highlighted. Optionally, the background may be subtracted by performing an image acquisition without administration of contrast agent, thereby providing the background to be subtracted from the image of the vascular system. This is referred to as digital subtraction angiography (DSA).

The contrasts resulting from this type of imaging of a vascular system are dependent on the imaging parameters, on characteristics of the examination subject, on a weight, for example, and on contrast agent administration parameters, specifically on how much contrast agent is used, for example, or the pressure with which it is injected, on the concentration of iodine or gadolinium in the contrast agent, on the viscosity of the contrast agent, on the mixing ratio of contrast agent and blood, and/or on the diameter of the injection catheter.

Various contrast enhancement methods are known. For example, the imaging of the vascular system may be subdivided into different frequency bands and the amplitudes of the frequency bands may be individually varied in order to accentuate frequency bands that are particularly relevant for the visualization of vessel details, whereas amplitudes of bands that primarily show noise or large background structures may be lowered. The scaling of the individual frequency bands may be predefined as fixed in advance or be dependent on the vessel imaging itself, specifically on its image data.

Despite the possibilities for improving contrast, there nonetheless remains the problem that because of the dependence of the resulting contrast on a large number of parameters it is hardly possible to compare the images of vascular systems of different patients. Even images acquired of the vascular system of the same patient are not comparable as a matter of course if for example different acquisition parameters or differing contrast agent injections are used.

If, for example, a predefined fixed algorithm is used for contrast optimization, low contrasts may result if, for example, an injection is performed at low pressure or with a small amount of contrast agent. If, on the other hand, adaptive approaches are used which take the image contents into account, differences may actually be hidden as a result. For example, an implanted stent may lead to a significantly higher blood flow in a part of the vascular system. However, changes in contrast produced as a result thereof may be eliminated again by the adaptive contrast optimization. In both cases, as also when non-contrast-enhanced images of the vascular system are used, only a very limited comparability is possible in respect of vascular system images that were acquired from different examination subjects, using different imaging parameters and/or with differences in contrast agent administration.

In order to counteract this problem, the publication WO 2019/002 560 A1 proposes taking imaging parameters used into account for the contrast enhancement. The publication EP 3 420 906 A1 furthermore proposes taking into account parameters of an automated contrast agent injection in the contrast enhancement. The two publications discussed use a relatively complex model for taking the cited parameters into account and therefore require relatively complicated calculations. Moreover, contrast agent administration parameters may be taken into account only if these are precisely known, that increases the overhead and is ultimately only possible in the case of an automatic contrast agent injection.

However, manual contrast agent injections are used in a large number of application cases, with the result that, for example, an exact pressure curve of the contrast agent injection is not known. In these cases, corresponding variations cannot be compensated for, thus continuing to make a comparison of different imaged vascular systems possible only to a limited extent.

### BRIEF SUMMARY AND DESCRIPTION

The scope of the present application is defined solely by the appended claims and is not affected to any degree by the statements within this summary. The present embodiments may obviate one or more of the drawbacks or limitations in the related art.

Embodiments include a method for processing x-ray images that provides a good comparability of vascular system images with low computational overhead or also in those cases in which a contrast agent injection is performed manually.

A computer-implemented method for processing x-ray images is provided that includes the following steps: providing a sequence of x-ray images of a field of view in which at least one section of a vascular system of an examination subject is imaged, wherein the sequence describes a variation with time of the detected x-ray intensity in the field of view based on a change in a contrast agent distribution in the section of the vascular system, determining a contrast agent variable relating to the contrast agent distribution as a function of a variation with time of a variable relating to the x-ray intensity in a selected subregion of the x-ray images, determining an image of the section of the vascular system as a function of at least one of the x-ray images of the sequence and modifying the image of the section of the vascular system as a function of the contrast agent variable in order to provide output data.

Embodiment include monitoring the variation with time of a variable relating to the x-ray intensity, for example a detected x-ray intensity or x-ray absorption, in a subregion of the x-ray image and inferring a contrast agent variable relating to the contrast agent distribution or contrast agent administration on the basis of the variation with time. In this process it is possible to exploit the knowledge that typically, within the scope of the image sequence, contrast agent is initially present only in a catheter or in a relatively small section of the vascular system, for example in that section of the vascular system for example via which the contrast agent flows into the field of view. Thus, even if initially there is no information available about the contrast agent administration parameters, inferences may be made from such a variation with time, for example from an increase in or decaying of the contrast agent concentration in the subregion and the associated variation in the x-ray intensity, concerning characteristics of the contrast agent administration and the contrasts that are to be expected therefrom.

The computer-implemented method is therefore well suited for processing sequences of x-ray images that were acquired during a manual administration of contrast agent since only incomplete information is available in respect of the contrast agent administration. Since modifying the image, including determining the contrast agent variable, as will be explained in more detail later, may be implemented by relatively simple algorithms in the method, the method may furthermore be carried out with low computational overhead.

The individual x-ray images may be two-dimensional x-ray images. However, it is also possible to use three-dimensional x-ray images that have been determined for example by a reconstruction method from a plurality of respective two-dimensional images. This may be beneficial, for example, to deploy the method in the field of computed tomography angiography. The individual x-ray images depict the same field of view in the same acquisition geometry in each case. This provides a subregion of the x-ray images, once it has been selected, to be located immediately in all of the x-ray images of the sequence, for example by taking the same pixels into account in each case.

The variable relating to the x-ray intensity may be for example a grayscale value of the respective pixel. Optionally, a preprocessing step may be performed, for example, to deduct offset values or similar. Absorption values may be used since absorption values may be directly correlated with a contrast agent concentration in the corresponding field of view. The sequence of x-ray images may be provided via an input interface and the output data may be output via an output interface. The input or output interfaces may be strictly software interfaces, though they may also be physical interfaces, for example network interfaces or interfaces of a data bus. The sequence of x-ray images may be provided for example by a database or similar. However, it is also possible for the sequence of x-ray images to be provided directly by a medical imaging device. This may be advantageous for example in order to use the computer-implemented method directly for evaluating the data of a corresponding image acquisition device.

The approaches known from the prior art that have already been explained in the introduction may be used for determining the imaging of the section of the vascular system. The problems described there in relation to the impact of different parameters on the resulting contrasts may be compensated for at least to a large extent by one or more embodiments.

An image section that images a catheter and/or a vessel via which the contrast agent was or is conducted into the imaged section of the vascular system in the course of the acquisition of the sequence, or a subsection of the image section, may be chosen as the selected subregion. Taking a subsection into account may serve to exclude the peripheral region of the image section from consideration. For example, the subsection may be a central section of the image section or include only a few central pixels of the image section. It is also possible that the subsection includes only a single pixel, for example a pixel located substantially centrally in the image section.

If the selected subregion includes multiple pixels, the variation with time may be determined for example for a minimum, a maximum, a median or a mean of the values of the pixels taken into account.

A variation with time of the variable relating to the x-ray intensity, for example an x-ray absorption, in a region may provide information in respect of the evolution over time in the concentration of the contrast agent in the region. By evaluating the contrast agent concentration or a variable dependent thereon in the region of a catheter or vessel via which contrast agent is conducted into the imaged region of the vascular system it is possible to take the variation with time of the contrast agent supply into account without the need for the contrast agent administration parameters, for example an injection quantity or an injection pressure, to be known for this.

The selection of the selected subregion may be dependent solely on one of the x-ray images or on a subsequence of the x-ray images including not all of the x-ray images. the x-ray image or subsequence may image the entry of the contrast agent into the imaged section of the vascular system.

For example, it may be one x-ray image or a subsequence of x-ray images that were acquired immediately after the contrast agent injection into the examination subject or immediately after the entry of the contrast agent into the imaged section of the vascular system. In such early x-ray images, the contrast agent is substantially present only in a catheter supplying the contrast agent or in a vessel via which the contrast agent is conducted into the imaged section of the vascular system. This results in a strong contrast for the region via which the contrast agent is conducted into the imaged section of the vascular system, thus providing the region to be easily segmented, for example, in order for it to be used as a subregion or in order for a subregion to be chosen within the region.

In order to select the selected subregion, a relevant image segment may be determined in which the image data in the single x-ray image or an intermediate image determined as a function of the subsequence is compared with a limit value. A high contrast agent concentration leads to a strong x-ray absorption and consequently to a low x-ray intensity. Regions with a high contrast agent concentration may therefore be segmented for example by choosing all interrelated pixels whose grayscale value or detected x-ray intensity lies below a limit value. If the variable relating to the x-ray intensity is chosen such that it increases with higher contrast agent concentration, specifically if an x-ray absorption is evaluated, for example, interrelated regions in which a limit value is exceeded may alternatively also be segmented.

The relevant segment may be the previously discussed image section that images a catheter and/or a vessel via which contrast agent was conducted into the imaged section of the vascular system in the course of the acquisition of the sequence. As explained above, this may be achieved by evaluation of an x-ray image or a subsequence of x-ray images that was acquired immediately after the administration of the contrast agent or the entry of the contrast agent into the imaged section of the vascular system. The selected subregion may be the relevant image segment or a subsegment of the relevant image segment. For example, a central region or an individual pixel within the relevant image segment may be selected for which the variation with time of the variable relating to the x-ray intensity is evaluated.

Determining an intermediate image may serve to identify all regions for which a high contrast agent concentration and consequently a low x-ray intensity is present in at least one of the x-ray images of the evaluated subsequence. This may be realized for example by setting the grayscale value of each pixel of the intermediate image to the smallest grayscale value for the pixel that occurs in the subsequence. This is beneficial if the grayscale value increases with the detected x-ray intensity. A minimum grayscale value therefore corresponds to the highest occurring contrast agent concentration. If an absorption or another variable that increases with increasing contrast agent concentration is already imaged as the variable describing the x-ray intensity in the individual x-ray images, then the maximum value that occurs for each pixel within the subsequence may be selected instead for the pixel.

The contrast agent concentration in the imaged section of the vascular system is additionally dependent on the diameter of a catheter used for supplying the contrast agent. The same applies when the contrast agent is supplied to the imaged sections of the vascular system via a vessel. It is therefore advantageous to take into account not only the x-ray attenuation resulting due to the contrast agent supply, and consequently substantially the integral over the contrast agent concentration perpendicular to the image plane, but also the diameter of the vessel or catheter via which the contrast agent is conducted into the imaged section of the vascular system. As will be explained in the following, such a diameter may be determined with good accuracy from the x-ray images themselves.

In the method, a diameter of the selected subregion or of the relevant image segment or of the or a vessel or catheter via which the contrast agent was or is conducted into the imaged section of the vascular system in the course of the acquisition of the sequence may be determined, the image of the section of the vascular system being modified as a function of the determined diameter. The diameter may be determined from image data or at least as a function of image data of at least one of the x-ray images or of a portion of the x-ray images of the sequence.

Possible segmentation options for the vessel or catheter used to supply the contrast agent have already been explained above. If the evaluated x-ray image or subsequence already depicts a penetration of the contrast agent into the vascular system over a certain length or if a certain length of the catheter is imaged, a longitudinal direction of the vessel or catheter may already be determined on the basis of the shape of the determined segment, with the result that the diameter may be determined for example as a dimension perpendicular to a determined longitudinal direction or as the shortest dimension.

As explained in the introduction, sequences of three-dimensional x-ray images may also be processed by the method. it is possible, as explained above, to take a diameter into account. However, a cross-sectional area of the subregion or of the relevant image segment or of the or a vessel or catheter may also be determined on the basis of the available three-dimensional x-ray image, whereupon the image of the section of the vascular system is modified as a function of the determined cross-sectional area.

In order to modify the images of the section of the vascular system, all image data of the image or that image data of the image that lies within a predefined value range may be multiplied, for example after deduction of an offset, by a scaling factor that is dependent on the contrast agent variable. For example, the entire scale of the image data may be compressed or stretched as a function of the contrast agent variable to increase or reduce the contrast. In some cases, however, it may also be advantageous to stretch or compress only a part of the value scale, for example, to achieve an increase in contrast selectively for specific ranges of grayscale values and, for example, to compress less relevant ranges of the scale.

In addition, or alternatively, the scaling factor may be dependent on the diameter or the cross-sectional area of the selected subregion or of the relevant image segment or of the or a vessel or catheter via which the contrast agent is supplied. The value range for which the scaling is to be performed and/or an offset may also be dependent on the contrast agent variable and/or the diameter or the cross-sectional area.

The scaling factor may be inversely proportional to the contrast agent variable and/or to the determined diameter. For example, it may be inversely proportional to a product from these two variables. In other words, the scaling factor may be proportional to the reciprocal of the contrast agent variable and/or to the reciprocal of the determined diameter, for example proportional to the reciprocal of the product from contrast agent variable and diameter.

Thus, a large scaling factor may result from a small contrast agent variable, that for example describes a small amount of contrast agent used or a lower supply pressure of the contrast agent, or from a small diameter of the catheter or vessel used to supply the contrast agent, as a result of which there is an increase in contrast in these cases, and vice versa. In this way, variations in the amount of contrast agent used, the type of contrast agent administration and the supply diameters for the contrast agent may be compensated for at least to a large extent by the choice of the scaling factor described. As the contrast agent variable may also depend on imaging parameters or parameters of the examination subject, deviations in this respect may also be compensated for at least to a large extent.

The contrast agent variable may be determined from the maximum or minimum of the variation with time of the variable relating to the x-ray intensity in the selected subregion or from an integral over the variation with time. In one case, the variable relating to the x-ray intensity correlates with an x-ray absorption and consequently at least approximately with a contrast agent concentration or the product from contrast agent concentration and the thickness of the region perfused by contrast agent perpendicular to the image plane. The maximum therefore occurs at the time of maximum contrast agent density.

If a relatively high flow rate is expected in the imaged section of the vascular system, it may at least approximately be assumed that the diffusion of the contrast agent bolus may be ignored, the detected maximum of the contrast agent concentration thus traveling through the vascular system. If the imaging of the section of the vascular system is performed as explained above in that maximally occurring absorptions or minimally occurring x-ray intensities are imaged for each pixel, then the contrast in the overall image therefore scales with the maximum of the variation with time, as a result of which the maximum represents a suitable contrast agent variable for the method. If the variable relating to the x-ray intensity relates for example directly to the detected x-ray intensity, then the above-explained maximum for the absorption corresponds to a minimum of the detected x-ray intensity.

If a relatively low flow rate is expected in the imaged section of the vascular system, the resulting contrast in the image of the section of the vascular system is primarily dependent on the total amount of contrast agent supplied. A not insubstantial portion of the contrast agent is dispersed in the vascular system due to a diffusion of the contrast agent, with the result that brief local maxima of the contrast agent concentration occurring during the supplying of the contrast agent, due for example to pressure fluctuations in the contrast agent supply, are scattered in the course of the propagation of the contrast agent into the vascular system.

If an x-ray absorption is used as the variable relating to the x-ray intensity, the integral over the variation with time is a good measure for the amount of contrast agent used for a given diameter or cross-sectional area of the supplying vessel or catheter. It is easy to recognize from the law of absorption that a corresponding variable may also be determined for example as a function of an integral of the logarithmic x-ray intensity.

One of a number of methods for determining the contrast agent variable may be selected as a function of a predicted flow rate and/or a duration of the contrast agent injection. At high flow rates or with short injection times, the maximum or minimum of the variation with time may be used, for example as explained above, to determine the contrast agent variable. A compact contrast agent bolus passes through the vascular system, as a result of which the total amount of injected contrast agent is rather less significant. If long injection times and/or slow flow rates are assumed, it may be advantageous, as explained above, to employ an integral-based approach for determining the contrast agent variable. It is likely that substantially the entire imaged section of the vascular system is flooded with contrast agent in the course of the sequence, with the result that occurring contrasts are primarily dependent on the total injected amount of contrast agent.

The flow rate or duration of the contrast agent injection may be predefined manually for a specific sequence or be dependent for example on an examined body region. Thus, it is known for example that much slower flow rates occur in the vascular system of the brain than in the peripheral regions of the body. However, it is also possible to obtain or at least estimate corresponding parameters from the image data itself. If, as explained above, a catheter or vessel has been identified via which the contrast agent is conducted into the imaged section of the vascular system in the course of the acquisition of the sequence, it may be determined, for example at different times of the sequence, how far the contrast agent has already penetrated into the vascular system, for example by determining whether limit values predefined for a specific x-ray image acquisition are exceeded or undershot by the variable relating to the x-ray intensity in specific sections of the vascular system. At least a rough estimation of the flow rate is possible.

The duration of the contrast agent injection may be determined for example by determining the x-ray image, and consequently a time instant, at which there is a steady decline in the contrast agent concentration in the supplying catheter or vessel or in a part of the vascular system immediately adjacent thereto.

The selection of the method used to determine the contrast agent variable and/or the determination of the contrast agent variable may also be dependent on other parameters. For example, a volume of the vascular system or at least of the imaged section of the vascular system or an area in the image occupied by the imaged section of the vascular system may be taken into account. The section may be segmented in the image of the section of the vascular system, for example on the basis of a limit value for a grayscale value, in order to obtain at least a rough estimate of the volume or area.

Embodiments also provide a processing device that is configured for performing the computer-implemented method.

Embodiments also provide an x-ray device for medical imaging that includes a processing device. The x-ray device may also include an acquisition device configured for capturing the x-ray images of the sequence, for example an x-ray source and an x-ray detector. The processing device may additionally be configured to capture the sequence by way of an acquisition device or to control the latter.

Alternatively, to the integration of the processing device into an x-ray device, the processing device may also be configured as a separate device. For example, the processing device may be a workstation computer, a local server, a server connected via a network, for example in a different building, or a cloud-based processing device for decentralized data processing.

Embodiments also provide a computer program for a data processing device, the program including program instructions that perform the computer-implemented method when the program is executed on the data processing device, and to a computer-readable data medium including such a computer program.

The processing device, the x-ray device, the computer program, and the computer-readable data medium may be developed by the features explained in relation to the computer-implemented method with the advantages cited there, and vice versa. Features disclosed in relation to individual embodiments may be applied in each case to the other embodiments.

### BRIEF DESCRIPTION OF THE FIGURES

FIG. 1 depicts a flowchart of an embodiment of a computer-implemented method according to an embodiment.

FIG. 2 depicts algorithms and data structures that may be used in an implementation of the method depicted in FIG. 1 according to an embodiment.

FIG. 3 depicts an x-ray image according to an embodiment.

FIG. 4 depicts a variation with time of a variable relating to the x-ray intensity in a selected subregion of x-ray images of a sequence according to an embodiment.

FIG. 5 depicts an image of a section of a vascular system according to an embodiment.

FIG. 6 depicts an embodiment of an x-ray device including an embodiment of a processing device.

### DETAILED DESCRIPTION

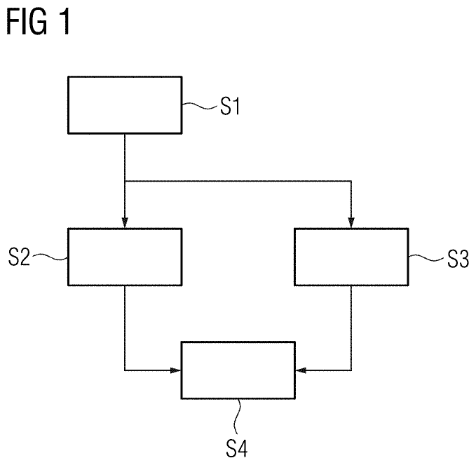

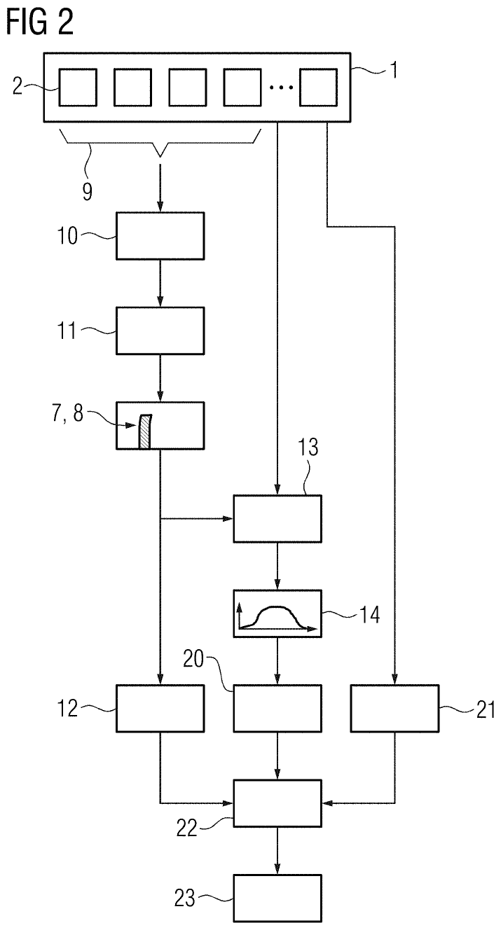

FIG. 1 depicts a flowchart of a computer-implemented method for processing x-ray images. Possible ways of implementing the individual method steps are explained in more detail here with reference to FIG. 2, that depicts algorithms and data structures that may be used to implement the method.

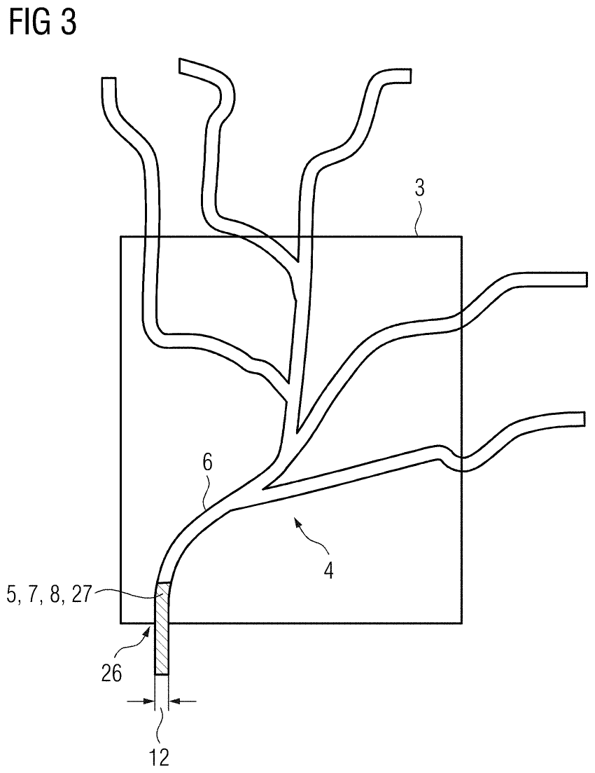

In step S1, a sequence 1 of x-ray images 2 of a field of view 3 depicted schematically in FIG. 3 is provided, at least one section of a vascular system 4 of an examination subject being imaged in the field of view 3. The sequence 1 describes a variation with time of the detected x-ray intensity in the field of view 3 due to a change in a contrast agent distribution in the section of the vascular system 4. In the example depicted in FIG. 3, the contrast agent is introduced into the field of view 3 from the side of the image edge 26 and the figure depicts an x-ray image that lies relatively early in the sequence, whereby contrast agent is present substantially exclusively in the vessel 27 via which the contrast agent is supplied to the vascular system 4, as a result of which an x-ray intensity in the image section 5 imaging the contrast agent is reduced or an absorption is increased. The remaining section 6 of the vascular system 4 is initially substantially free of contrast agent and consequently produces only low image contrasts.

The sequence 1 may be provided via an input interface and be read out from a database, for example. However, it is also possible to deliver the sequence by providing and processing the x-ray images immediately after they have been acquired.

In step S2, a contrast agent variable 20 relating to the contrast agent distribution is determined as a function of a variation with time 14 of a variable relating to the x-ray intensity in a selected subregion 7 of the x-ray images 2. To that end, the subregion 7 is initially selected in such a way that it lies in a region of an imaged catheter or vessel 27 via which the contrast agent is conducted into the imaged section of the vascular system 4 in the course of the acquisition of the sequence 1. For this purpose, the relevant image segment 8 in an x-ray image 2 might be segmented at the start of the sequence 1. This approach may be beneficial, for example, when it is known that a catheter via which contrast agent is supplied lies within the field of view 3, such that it is ensured that a region filled with contrast agent is already present in these early x-ray images 2.

If the contrast agent is introduced into the vascular system 4 from outside of the field of view 3, it is possible that no contrast agent will yet be imaged on individual images of the x-ray images 2. In order to ensure that a vessel 27 or catheter supplying the contrast agent may be robustly identified, it may therefore be advantageous to use a subsequence 9 of x-ray images 2 initially, for example at the start of the sequence 1, in order to generate an intermediate image 10. In this instance, for each pixel of the intermediate image 10, the value of the corresponding pixel of that image of the x-ray images 2 of the subsequence 9 may be chosen in each case that corresponds to the lowest received x-ray intensity or, as the case may be, the highest absorption. In this way it may be provided that the highest occurring contrast agent concentration within the subsequence 9 is imaged in each case in the intermediate image 10.

The intermediate image 10 is subsequently segmented by a segmentation algorithm 11 to determine the image section 5 in which the vessel 27 via which the contrast agent was conducted into the imaged section of the vascular system 4 in the course of the acquisition of the sequence 1 is imaged. The image data in the intermediate image 10 is compared with a limit value in order to determine a relevant image segment 8 that corresponds to the image section 5 that is being sought. This entails searching for a region containing contrast agent, specifically a region in which a particularly high absorption or a particularly low detected x-ray intensity is present. Thus, if the image data describes a detected x-ray intensity, a coherent segment in which the image data undershoots a predefined limit value may be chosen as the relevant image segment. If an x-ray absorption is described by the image data, a coherent relevant image segment 8 may be chosen in which the predefined limit value is exceeded for all pixels, specifically a particularly high absorption is present.

The entire relevant image segment 8 may be taken into account as the selected subregion 7. Alternatively, it would also be possible to choose only a part of the relevant image segment 8, for example a central region, specifically one or more pixels located for example centrally in the relevant image segment 8, as the selected subregion 7.

A variation with time 14 of a variable relating to the x-ray intensity is subsequently determined by the evaluation algorithm 13 for the thus chosen subregion. For example, an x-ray absorption or a variable proportional thereto may be used as the variable. The x-ray absorption is indirectly proportional to the logarithm of the x-ray intensity incident on the detector as far as an at least approximately constant emitted x-ray dose may be assumed.

The variation with time 14 is determined in that the evaluation algorithm 13 takes into account in each case, for each of the x-ray images 2 of the sequence 1, the pixels that lie in the previously determined selected subregion 7, specifically for example in the relevant image segment 8. A respective value of the variable relating to the x-ray intensity is determined for each of the pixels. Since the x-ray images 2 are acquired in chronological sequence and therefore overall describe a variation with time of the x-ray intensity in each pixel of the field of view 3, the variables relating to the x-ray intensity that were determined for the individual x-ray images 2 again result in a variation with time of the variable relating to the x-ray intensity in the selected subregion.

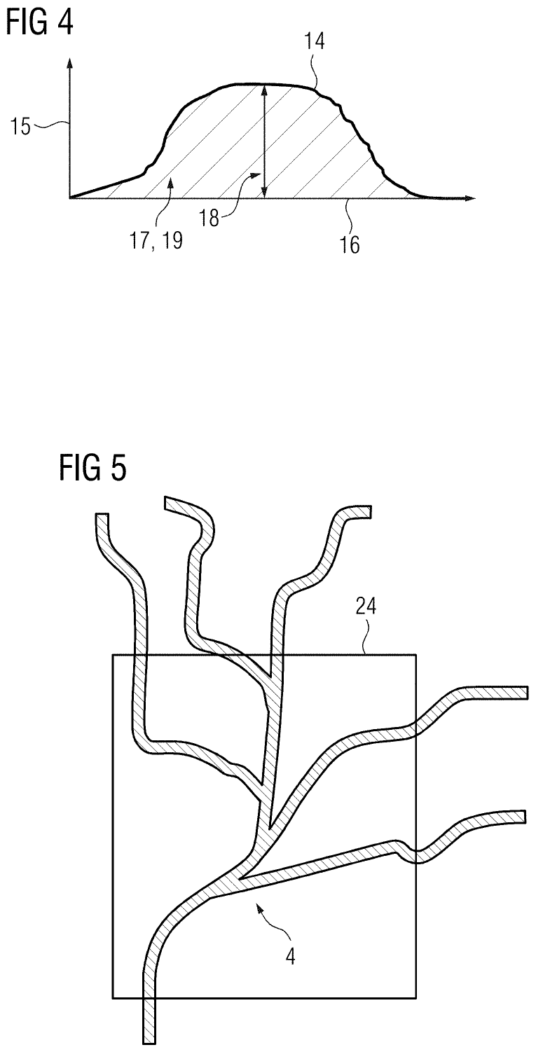

An embodiment with time 14 is depicted in FIG. 4 for the case in which the variable relating to the x-ray intensity is proportional to the x-ray absorption and consequently substantially to the contrast agent concentration in the selected subregion 7. Here, the X-axis 16 depicts the time and the Y-axis 15 the value of the variable. At the start of the contrast agent injection, the concentration of the contrast agent in the selected subregion 7 is initially still relatively low since sufficient contrast agent has not yet entered the imaged field of view 3. With a typical contrast agent injection, the contrast agent concentration in the selected subregion 7, and consequently the x-ray absorption, as shown in FIG. 4, will increase relatively quickly over time and after a certain time, specifically, for example, after the end of the contrast agent injection, will decrease again. This results in the variation with time 14 depicted in FIG. 4.

As already explained, given a relatively high expected flow rate in the imaged section of the vascular system 4, for example the maximum concentration of the contrast agent achieved in the selected subregion, and consequently the maximum 18 of the variation with time 14, is relevant for the contrast of the vascular system. The value of the variable at the maximum 18 may therefore be determined as the contrast agent variable.

As likewise already explained, with long injection times and/or at slow flow rates, how much contrast agent has been injected overall may also be important for the contrast in the image 21, 24 of the section of the vascular system 4. Since the amount of contrast agent that is present in the selected subregion 7 at the time of acquisition of the respective x-ray image 2 correlates in the explained example with the x-ray absorption, and consequently with the variable relating to the x-ray intensity, the integral 17 of the variation with time 14 of the variable, specifically the area 19 below the curve describing the variable, may be used as the contrast agent variable 20.

In step S3, an image 21, 24 of the vascular system 4 is determined as a function of the x-ray images 2 of the sequence 1, as is depicted by way of example in FIG. 5. The image 21, 24 may be generated by choosing, for each pixel of the image 21, 24, the value of the associated pixel of that image of the x-ray images 2 that corresponds to the lowest detected x-ray intensity or, as the case may be, to the highest absorption, and consequently to the highest contrast agent concentration in the region of the pixel. This provides an optimal imaging of the vascular system 4.

In order to make a comparability of the images 21, 24 of the same vascular system 4 or of different vascular systems 4 easier, a modification of the image 21, 24 for contrast correction is performed in step S4. For this purpose, the image 21, 24 is modified as a function of the contrast agent variable 20 in order to provide output data 23.

As has already been explained, a high contrast agent variable 20 may correlate with a high contrast in the image 21, 24. Consequently, an increase in contrast should be affected for small contrast agent variables 20 and potentially a reduction in contrast for large contrast agent variables 20 in order to achieve a uniform image impression. In other words, the image data of the image 21, 24, or at least that image data of the image 21, 24 that lies within a predefined value range within which it is aimed to adjust the contrast, may be scaled or multiplied with a scaling factor that is inversely proportional to the contrast agent variable.

The diameter 12 (shown in FIG. 3) of the vessel 27 or catheter via which the contrast agent is supplied likewise influences the contrast of the image 21, 24. larger diameters 12 lead to stronger contrasts in the image 21, 24. For this reason the diameter 12 may be determined in addition for example by determining a minimum dimension of the relevant image segment 8. The scaling factor used in the course of the modification 22 may be inversely proportional to the product from the determined contrast agent variable 20 and the determined diameter 12. The image data of the image 21, 24 may be divided, for example after deduction of an offset, both by the determined contrast agent variable 20 and by the determined diameter 12 and subsequently multiplied for example by a predefined fixed factor.

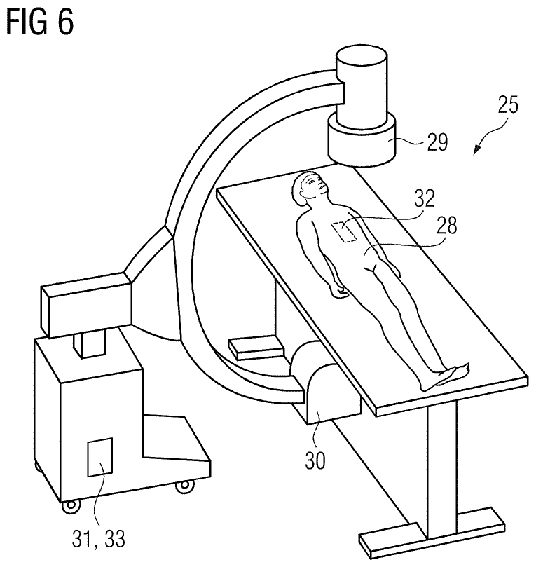

FIG. 6 depicts an x-ray device 25 for medical imaging. X-ray radiation provided by an x-ray source 29 is passed through an examination subject 28 and the x-ray radiation that has passed through the examination subject 28 is detected by the x-ray detector 30. A portion of the emitted x-ray radiation is absorbed by the examination subject 28 as well as by a contrast agent (not shown) introduced into the vascular system of the examination subject 28, as a result of which the contrast agent distribution in the examination subject 28 is identifiable in the x-ray images captured by the x-ray detector 30. If a sequence of x-ray images of the region 32 of the examination subject 28 is acquired using the same acquisition geometry during an administration of contrast agent, the sequence of x-ray images may be processed by the previously explained computer-implemented method in order to obtain a contrast-adjusted image of a section of the vascular system of the examination subject 28.

A processing device 31, that in the example shown is integrated into the x-ray device 25 but might also be configured as a separate processing device, implements the explained computer-implemented method. The processing device 31 may be implemented for example by appropriate programming of a programmable data processing device 33, in which case the data processing may be carried out for example by a microprocessor, microcontroller, FPGA or similar. The processing device may be additionally configured to control the x-ray source 29 for the acquisition of the sequence of x-ray images and to capture the corresponding x-ray images via the x-ray detector 30.

In a case not depicted, where the processing device is configured separately from the x-ray device, the same may be implemented for example by a workstation computer, for example for image data evaluation, by a local server or a server connected via a network and/or non-centrally, for example as a cloud solution.

It is to be understood that the elements and features recited in the appended claims may be combined in different ways to produce new claims that likewise fall within the scope of the present application. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present application has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for processing x-ray images, the method comprising:
providing a sequence of x-ray images of a field of view in which at least one section of a vascular system of an examination subject is imaged, wherein the sequence of x-ray images describes a variation with time of a detected x-ray intensity in the field of view based on a change in a contrast agent distribution in the section of the vascular system;
determining a contrast agent variable relating to the contrast agent distribution as a function of a variation with time of a variable relating to the detected x-ray intensity in a selected subregion of the sequence of x-ray images;
determining an image of the at least one section of the vascular system as a function of at least one x-ray image of the sequence of x-ray images; and
modifying the image of the at least one section of the vascular system as a function of the contrast agent variable to provide output data.

2. The computer-implemented method of claim 1, wherein the selected subregion comprises an image section that images a catheter, a vessel, or the catheter and the vessel via which the contrast agent was or is conducted into the imaged section of the vascular system during an acquisition of the sequence or a subsection of the image section.

3. The computer-implemented method of claim 1, wherein the selection of the selected subregion is dependent solely on one x-ray image of the sequence of x-ray images or on a subsequence of x-ray images comprising not all of the x-ray images of the sequence of x-ray images, wherein the one x-ray image or subsequence of x-ray images include an entry of the contrast agent into the imaged section of the vascular system.

4. The computer-implemented method of claim 3, wherein in order to select the selected subregion, a relevant image segment is determined in that image data in the one x-ray image or in an intermediate image determined as a function of the subsequence is compared with a limit value.

5. The computer-implemented method of claim 1, further comprising:
determining a diameter of the selected subregion or of a relevant image segment or of a vessel or a catheter via which the contrast agent was or is conducted into the imaged section of the vascular system during acquisition of the sequence of x-ray images, wherein the image of the section of the vascular system is modified as a function of the determined diameter.

6. The computer-implemented method of claim 1, wherein that in order to modify the image of the section of the vascular system, all image data of the image or image data of the image that lies within a predefined value range may be multiplied after deduction of an offset by a scaling factor that is dependent on the contrast agent variable.

7. The computer-implemented method of claim 6, wherein the scaling factor is inversely proportional to the contrast agent variable, the determined diameter, or the contrast agent variable and the determined diameter.

8. The computer-implemented method of claim 1, wherein the contrast agent variable is determined from the maximum or minimum of the variation with time of the variable relating to the x-ray intensity in the selected subregion or from an integral over the variation with time.

9. The computer-implemented method of claim 1, wherein one of a number of methods for determining the contrast agent variable is selected as a function of a predicted flow rate, a duration of a contrast agent injection, or the predicted flow rate and the duration of the contrast agent injection.

10. An x-ray system for medical imaging, comprising:
an acquisition device configured for capturing a sequence of x-ray images of a field of view in which at least one section of a vascular system of an examination subject is imaged, wherein the sequence of x-ray images describes a variation with time of a detected x-ray intensity in the field of view based on a change in a contrast agent distribution in the at least one section of the vascular system; and
a processing device configured to determine a contrast agent variable relating to the contrast agent distribution as a function of a variation with time of a variable relating to the detected x-ray intensity in a selected subregion of the sequence of x-ray images, determine an image of the at least one section of the vascular system as a function of at least one x-ray image of the sequence of x-ray images, and modify the image of the at least one section of the vascular system as a function of the contrast agent variable to provide output data.

11. The x-ray system of claim 10, wherein the selected subregion comprises an image section that images a catheter, a vessel, or the catheter and the vessel via which the contrast agent was or is conducted into the imaged section of the vascular system during acquisition of the sequence or a subsection of the image section.

12. The x-ray system of claim 10, wherein the selection of the selected subregion is dependent solely on one x-ray image or on a subsequence of the x-ray images comprising not all of the x-ray images of the sequence of x-ray images, wherein the one x-ray image or subsequence of x-ray images include an entry of the contrast agent into the imaged section of the vascular system.

13. The x-ray system of claim 12, wherein in order to select the selected subregion, a relevant image segment is determined in that image data in the one x-ray image or in an intermediate image determined as a function of the subsequence is compared with a limit value.

14. The x-ray system of claim 10, wherein the processing is further configured to determine a diameter of the selected subregion or of a relevant image segment or of a vessel or a catheter via which the contrast agent was or is conducted into the imaged section of the vascular system during acquisition of the sequence, wherein the image of the section of the vascular system is modified as a function of the determined diameter.

15. The x-ray system of claim 10, wherein that in order to modify the image of the section of the vascular system, all image data of the image or image data of the image that lies within a predefined value range may be multiplied after deduction of an offset by a scaling factor that is dependent on the contrast agent variable.

16. The x-ray system of claim 15, wherein the scaling factor is inversely proportional to the contrast agent variable, the determined diameter, or the contrast agent variable and the determined diameter.

17. The x-ray system of claim 10, wherein the contrast agent variable is determined from the maximum or minimum of the variation with time of the variable relating to the x-ray intensity in the selected subregion or from an integral over the variation with time.

18. The x-ray system of claim 10, wherein one of a number of methods for determining the contrast agent variable is selected as a function of a predicted flow rate, a duration of a contrast agent injection, or the predicted flow rate and the duration of the contrast agent injection.

19. A non-transitory computer implemented storage medium that stores machine-readable instructions executable by at least one processor, the machine-readable instructions comprising:
acquiring a sequence of x-ray images of a field of view in which at least one section of a vascular system of an examination subject is imaged, wherein the sequence of x-ray images describes a variation with time of the detected x-ray intensity in the field of view based on a change in a contrast agent distribution in the at least one section of the vascular system;
determining a contrast agent variable relating to the contrast agent distribution as a function of a variation with time of a variable relating to the x-ray intensity in a selected subregion of the sequence of x-ray images;
determining an image of the at least one section of the vascular system as a function of at least one of the x-ray images of the sequence of x-ray images; and
modifying the image of the at least one section of the vascular system as a function of the contrast agent variable to provide output data.
